(19) United States
(12) Patent Application Publication
(10) Pub. No.: US 2026/0324970 A1
(43) Pub. Date: Oct. 22, 2026

(54) OPTICAL ATTACHMENTS AND METHODS FOR EMITTING LIGHT

(71) Applicant: ICARE FINLAND OY, Vantaa (FI)

(72) Inventor: Teemu Herranen, Hyvinkää (FI)

(73) Assignee: ICARE FINLAND OY, Vantaa (FI)

(21) Appl. No.: 19/491,085

(22) PCT Filed: Apr. 17, 2024
(86) PCT No.: PCT/FI2024/050179

§ 371 Date: Dec. 8, 2025

(30) Foreign Application Priority Data
Jun. 30, 2023 (FI) 20235775

(51) Int. Cl. A61B 3/14; A61B 3/16

(52) U.S. Cl. CPC A61B 3/14; A61B 3/16

(57) ABSTRACT

An optical attachment for emitting light towards a target, the optical attachment including a hollow body of light guiding material, the hollow body including a through opening, an outer surface, an inner surface opposite to the outer surface, a first output facet, and a posterior facet opposite to the first output facet; and the posterior facet arranged to receive light from one or more light sources and to guide the received light via the hollow body towards the first output facet; and wherein the first output facet when in use is configured to emit light towards the target forming a first illuminated image of the first output facet on the target.

### TECHNICAL FIELD

The present disclosure relates to optical attachments for emitting light towards a target. The present disclosure also relates to methods of emitting light towards a target.

### BACKGROUND

Commonly, living beings (such as, for example, human beings) suffer from eye ailments, such as, but not limited to, glaucoma, ocular hypertension, retinal detachment. Such eye ailments can be determined by determining at least one physiological property of the eye using tonometry methods. The tonometry methods are implemented using specialized equipment such as, for example, a tonometer, to measure at least one physiological property of the eye. An example of the at least one physiological property may be a pressure within the eye, wherein when the pressure exceeds a predefined range of pressure, an optic nerve of the eye may get damaged. This causes vision loss of the eye.

Existing measurement techniques to measure the physiological properties of the eye suffer from certain limitations. Conventional tonometry method implements a tonometer to measure the at least one physiological property of the eye of a patient, wherein the tonometer comprises a magnetic probe for measuring the at least one physiological property. Additionally, the magnetic probe has a round tip, which when detachably attaching from/to the tonometer, can be accidentally touched by an operator of the tonometer, thereby increasing the risk of infection. The magnetic eye probe also needs to be aligned at a predefined distance from the eye in order to hit a particular area of a surface of the eye multiple times for accurate and precise measurements. However, due to any accidental change in position of the patient or of the tonometer during the measurement of the at least one physiological parameter, the magnetic probe may hit another area of the surface of the eye, thereby providing inaccurate measurements. Further more it is important to perform measurements at a correct distance from a surface of the eye (not too close nor too far away).

Therefore, in light of the foregoing discussions, there exists a need to overcome the aforementioned drawbacks associated with conventional tonometer methods for measuring the at least one physiological parameter of the eye.

### SUMMARY

The present disclosure seeks to provide an optical attachment for emitting light towards a target. The present disclosure also seeks to provide a method for emitting light towards a target. An aim of the present disclosure is to provide a solution that overcomes at least partially the problems encountered in prior art.

In one aspect, an embodiment of the present disclosure provides an optical attachment for emitting light towards a target, the optical attachment comprising:
a hollow body of light guiding material, the hollow body comprising a through opening, an outer surface, an inner surface opposite to the outer surface, a first output facet, and a posterior facet opposite to the first output facet; and
the posterior facet arranged to receive light from one or more light sources and to guide the received light via the hollow body towards the first output facet; and
wherein the first output facet when in use is configured to emit light towards the target forming a first illuminated image of the first output facet on the target.

In another aspect, an embodiment of the present disclosure provides a method of determining a distance between an surface of the eye and a tonometer, the method comprising using an optical attachment according to provide a first illuminated image of a first output facet of the optical attachment on the surface of the eye. Embodiments of the present disclosure substantially eliminate or at least partially address the aforementioned problems in the prior art, and enable an efficient and specialised equipment for emitting light towards the target, to properly align the optical attachment with respect to the target.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIGS. 1A and 1B show schematic illustrations of formation of a first illuminated image and a second illuminated image of an optical attachment for emitting light towards a target, in accordance with different embodiments of the present disclosure;

FIG. 2 shows an isometric view and a front view of an optical attachment, in accordance with an embodiment of the present disclosure;

FIG. 3 shows a schematic illustration of formation of a first illuminated image and a second illuminated image on a target, in accordance with an embodiment of the present disclosure;

FIGS. 4A, 4B, and 4C show exemplary schematic illustrations of formation of a first illuminated image and a second illuminated image on a target using an optical attachment, in accordance with an embodiment of the present disclosure;

FIGS. 5A and 5B is a cross-section view of an optical attachment when in use and connected to a tonomenter, in accordance with an embodiment of the present disclosure;

FIG. 6 shows an optical attachment comprising a detachable tube, in accordance with an embodiment of the present disclosure;

FIG. 7 illustrates a flowchart depicting steps of a method of emitting light towards a target using an optical attachment comprising a hollow body of a light guiding material, in accordance with an embodiment of the present disclosure;

FIGS. 8A and 8B are illustrations of how convex mirror analogy can be used in accordance with an embodiment of the present disclosure;

FIGS. 9A, 9B, and 9C are illustrations on how distance of the tonometer impacts size of reflected image; and

FIG. 10 is an illustration on point of view of person operating tonometer.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In a first aspect, an embodiment of the present disclosure provides an optical attachment for emitting light towards a target, the optical attachment comprising:
a hollow body of light guiding material, the hollow body comprising a through opening, an outer surface, an inner surface opposite to the outer surface, a first output facet, and a posterior facet opposite to the first output facet; and
the posterior facet arranged to receive light from one or more light sources and to guide the received light via the hollow body towards the first output facet; and
wherein the first output facet when in use is configured to emit light towards the target forming a first illuminated image of the first output facet on the target.

In a second aspect, an embodiment of the present disclosure provides a method of determining a distance between an surface of the eye and a tonometer, the method comprising using an optical attachment according to any of claim 1-12 to provide a first illuminated image of a first output facet of the optical attachment on the surface of the eye. The present disclosure provides the aforementioned optical attachment and the aforementioned method. Pursuant to the embodiments of the present disclosure, the optical attachment is optically efficient due to the light guiding material of the hollow body, wherein the light guiding material provides an optical path to the emitted light. Hence, the optical attachment functions as a light guiding system, which aligns the optical attachment at a required distance from the target. The required distance from the target can be observed by user of a measurement device (such as tonometer) visually. In an example a light forms a first illuminated image of the first output facet on the target (such as a surface of the eye). According to embodiments the first output facet is dimensioned in such a way the first illuminated image, which is formed at the target, corresponds to some relative size of detail of the target. In one embodiment the target is a surface of the eye and the first output facet is circular ring like. In said example a circular ring like first illuminated image is formed on the surface of the eye. If the circular first illuminated image as approximately size (such as for example 0.8 to 1.2 times radius of pupil) of pupil the device is a the right distance. Beneficially, such an optical attachment is easy to manufacture, as it does not require any advanced manufacturing processes. The optical attachment is simple, light-weight, robust, reliable and can be implemented with ease.

A shape of the hollow body is any one of: a hollow cylinder, a hollow circular truncated cone, a frustum of a cone. In deed the hollow body is, according to an embodiment, shaped as a conical frustum of a cylinder, wherein the first output facet forms a first base of the conical frustum, the posterior facet forms a second base the conical frustum and the first base of the conical frustum has a first radius. The light guiding material of the hollow body enables a passage of light therethrough. Examples of the light guiding material may include, but are not limited to, polypropylene material, polycarbonate material, polymethyl methacrylate (PMMA) material. Optionally, the outer surface of the optical attachment faces outward, and the inner surface of the optical attachment faces inward towards a centre of the hollow body. Optionally, the first output facet is a curved plane surface on an outer side of the hollow body proximal to the target, wherein the curved plane surface is formed between the outer surface and the inner surface. Herein, the curved plane surface is any one of a convex surface or a concave surface resembling a back surface of a hard contact lens. As an example, the first radius of the first output facet may be between 3, 4, 5, 6, 7, 8 up to 4, 5, 6, 7, 8, 9 or 10 millimetres As an example it can be 4 mm.

Optionally, the posterior facet is a plane surface opposite to the first output facet that is distal to the target, wherein the plane surface is formed between the outer surface and the inner surface. The term “through opening” refers to an opening in the centre of the hollow body which is formed based on a shape of the inner surface. In an instance, the outer surface is at an angle from the posterior facet towards the first output facet in such a manner that the outer surface of the hollow body tapers from the posterior facet towards the first output facet. In another instance, the outer surface is at an angle from the posterior facet towards the first output facet in such a manner that the outer surface of the hollow body widens from the posterior facet towards the first output facet. In yet another instance, the outer surface is at an angle from the posterior facet towards the first output facet in such a manner that the outer surface of the hollow body lies straight from the posterior facet towards the first output facet. Similarly, in an instance, the inner surface is at an angle from the posterior facet towards the first output facet in such a manner that the inner surface of the hollow body tapers from the posterior facet towards the first output facet. In another instance, the inner surface is at an angle from the posterior facet towards the first output facet in such a manner that the inner surface of the hollow body widens from the posterior facet towards the first output facet. In yet another instance, the inner surface is at an angle from the posterior facet towards the first output facet in such a manner that the inner surface of the hollow body lies straight from the posterior facet towards the first output facet.

Optionally, the hollow body is configured to surround an eye probe comprised in a tonometer. The term “tonometer” refers to an optical device that is used to measure an intraocular pressure (IOP) of an eye, when the target is the eye. Herein, the term “optical device” refers to an instrument that is used to measure at least one property of an eye, wherein the at least one property of the eye may be a physiological parameter concerning the eye such as, intra-ocular pressure of the eye, touch sensitivity and similar. The term “eye probe” refers to an instrument that is used for measuring the IOP of the eye. In one example the eye probe comprises a magnetised steel wire shaft covered with a tip (such as, for example, a round tip). In this regard, the tonometer processes a movement of the eye probe resulting from an interaction of the eye probe with the eye and is used for accurate measurement of the IOP of the eye. In an instance, the eye probe is detachably attached to the tonometer. In such an instance, the eye probe is a disposable eye probe. In other words, the disposable eye probe is intended for single use only and then discarded, to reduce a risk of infection transmission. In another instance, the eye probe is permanently attached to the tonometer. In such an instance, the eye probe is always in a same position relative to the tonometer, thereby ensuring consistent and accurate measurements.

Optionally, the optical attachment is arranged on the tonometer using the posterior facet of the hollow body of the optical attachment for being physically supported. Herein, the posterior facet of the optical attachment is any one of: detachably attached, permanently attached to the tonometer. In an instance, when the optical attachment is detachably attached to the tonometer, it means that the optical attachment is removable. In such an instance, the optical attachment can detach from the tonometer and be attached and used with another optical device. In another instance, when the optical attachment is permanently attached to the tonometer, it means that the optical attachment cannot be detached from the tonometer.

Optionally, the hollow body is configured to surround the eye probe in an unused state. A technical benefit of configuring the hollow body to surround the eye probe in an unused state is to provide a protection to the eye probe from an external environment and maintain a sterility of said eye probe. Another technical benefit is to provide protection to the eye probe from accidentally touching said eye probe, wherein the accidental touching may be caused by involuntary blinking of the eye. Yet another technical effect is to protect a movement of the eye probe. In other words, the hollow body protects the eye probe when said eye probe undergoes a rebound motion during the IOP measurement, thereby rendering the rebound motion error-free.

Optionally, the optical attachment further comprises a detachable tube configured to be accommodated inside the through opening, wherein the detachable tube is configured to accommodate the eye probe for attaching or detaching the eye probe to the tonometer. In this regard, a diameter of the detachable tube is smaller than a diameter of the inner surface of the first output facet. Furthermore, a length of the detachable tube is greater than or equal to a length of the eye probe. Moreover, the length of the detachable tube is greater than a length of the optical attachment. The detachable tube is a hollow cylindrical tube to contain the eye probe within. When attaching the eye probe to the tonometer, the detachable tube containing the eye probe is inserted into the through opening of the optical attachment to attach the eye probe to the tonometer via the posterior facet of the hollow body. Subsequently, when the eye probe is attached to the tonometer, the detachable tube is ejected, and the eye probe is surrounded by the hollow body of the optical attachment. In other words, a combination of the optical attachment and the detachable tube functions as a fixed probe applicator for the eye probe, wherein the eye probe is safely dropped into the through opening and detachably attached to the tonometer. When detaching the eye probe from the tonometer, the detachable tube is inserted into the through opening of the optical attachment. Subsequently, the eye probe is ejected into the detachable tube and the detachable tube containing the eye probe is detached from the tonometer. A technical benefit of configuring the detachable tube to accommodate the eye probe is to prevent any accidental contact of the tip of the eye probe when handling the tonometer while attaching or detaching the eye probe to the tonometer. Another technical effect is to easily switch out different eye probes, or to replace a damaged or malfunctioning eye probe using the detachable tube without having to replace the entire optical attachment. Beneficially, this enables the tonometer to be more versatile, and is time-efficient and cost-efficient.

Throughout the present disclosure, the term “light source” refers to an element from which light emanates. In other words, light source, when activated, emits light. Optionally, the one or more light sources comprises one or more of visible-light emitting diodes. Optionally, the one or more light sources are active light sources. This means the one or more light source provide the light by emitting the light themselves, wherein the light can be visible-light or near-infrared light. An example of the one or more light sources may include light-emitting diodes (LED).

Optionally, the one or more light sources are operable to generate different light colours or patterns indicative of different operative states of the tonometer. Herein, the different operative states of the optical device may include, but are not limited to, turning the tonometer on, attaching the eye probe to the tonometer, commencement of magnetization of the eye probe, conclusion of the magnetization of the eye probe, readiness of the tonometer for IOP measurement, detaching the eye probe from the tonometer. A technical benefit of generating different light colours or patterns is to create a variety of visual effects, such as for example, generating red colour in a pulsating manner may indicate incorrect attachment of the eye probe to the tonometer, or a green colour, when the eye probe is at a predefined distance for carrying out a measurement.

Optionally, the one or more light sources are electrically coupled to a driving circuit to provide an electrical connection between the one or more light sources and the driving circuit. In this regard, the driving circuit is responsible for controlling an operation of the one or more light sources. The term “driving circuit” refers to an electronic circuit that is responsible for controlling various aspects (such as, for example, turning the one or more light sources on or off) of the operation of the one or more light sources. Herein, the various aspects of the operation can be controlled by controlling a voltage provided to the driving circuit. The driving circuit receives input signals from a control system, which may be user-defined, automatic, or a combination of both. For example, the driving circuit may be designed to provide a given voltage to turn on two out of five light sources of the optical attachment.

Optionally, the one or more light sources are arranged on the optical device in a spatial manner, such that the light emitted from the one or more light sources is received by the first output facet. Alternatively, optionally, the one or more light sources are arranged on the posterior facet of the hollow body in a spatial manner, such that the light emitted from the one or more light sources is received by the first output facet. The shapes of the outer surface and the inner surface guides the received light via the hollow body towards the first output facet. For example, when the outer surface and the inner surface are tapered from the posterior surface towards the first output facet, the received light is guided is slanted based on an angles of tapering of the outer surface and the inner surface.

Optionally, the one or more light source are operable to generate stray light that obscures visibility of the eye probe during measurement of the at least one property of the eye. Herein, the term “stray light” refers to scattered light emitted by the one or more light sources via the outer surface of the optical attachment. When the optical attachment is in use and the tonometer is used for measuring the at least one property of the eye of a patient, the stray light is visualised as a fuzzy cone of light in front of the eye. This fuzzy cone of light obscures the visibility of the tip of the eye probe (due to contracst), as the eye of the patient can not see clearly the probe (or tip of the probe). In other words, the optical attachment functions as a fixation point for the patient, during measurement of the at least one property of the eye. A technical benefit of generating stray light is that the eye stays stable when looking on the stray light, hence the eye probe hits at a right angle on a surface of the eye for repeated measurement of the at least one property of the eye.

Throughout the present disclosure, the term “first illuminated image” refers to an image of the first output facet formed on the target, when the received light is emitted towards the target from the first output facet.

In other words, the first illuminated image of the first output facet is formed on the target when the light received from the one or more light sources is projected through the posterior surface of the hollow body to the target via the first output facet. In this regard, when in use, the first illuminated image represents a circular shape of the first output facet. When the optical attachment is in use, the formation of the first illuminated image provides an indication of a required distance of the optical attachment from the target. A technical benefit of forming the first illuminated image is that it is possible to define by the first illuminated image, whether the optical attachment is at a predefined distance (i.e., a safe distance or at operating distance) from the target. As an example, the first illuminated can be a circular ring like image. When the circular ring like image has approximately same size as pupil (or iris for example) then we can conclude that the device is at right distance.

The technical effect of the arrangement of the posterior facet and use of the first output facet is that it provides an accurate alignment of the optical attachment (namely, optical device). In this regard, the posterior facets guide the received light towards the target (for example, the eye's surface). The posterior facets enable the creation of a distinct illuminated image on the target, for instance by creating the illuminated image on the eye, which assists healthcare workers as they can see based on the illuminated image, whether the optical attachment is aligned correctly. Additionally, beneficially, the illuminated images are formed on the eye, so that a healthcare worker can determine whether the optical attachment is aligned correctly based on the images formed on the eye.

Optionally, the inner surface includes a stepped configuration defining a first portion, a second portion, and a second output facet between the first portion and the second portion, the second output facet being parallel to the first output facet, and
wherein the posterior facet is further arranged to guide the received light via the hollow body towards the second output facet, and
wherein the second output facet when in use is configured to emit the light towards the target forming a second illuminated image of the second output facet on the target.

In this regard, the second output facet is plane surface in an inner side of the hollow body between the first output facet and the posterior output facet. As an example, a radius of the second output facet may be 0.5, 1, 2 or millimetres smaller than radius of the first output facet. Herein, the first portion could be at an angle same as or different from an angle of the second portion. In an instance, the first portion is at an angle from the posterior facet towards the second output facet in such a manner that the first portion tapers from the posterior facet towards the second output facet. In another instance, the first portion is at an angle from the posterior facet towards the second output facet in such a manner that the first portion widens from the posterior facet towards the second output facet. In yet another instance, the first portion is at an angle from the posterior facet towards the second output facet in such a manner that the first portion lies straight from the posterior facet towards the second output facet. Similarly, in an instance, the second portion is at an angle from the second output facet towards the first output facet in such a manner that the second portion tapers from the second output facet towards the first output facet. In another instance, the second portion is at an angle from the second output facet towards the first output facet in such a manner that the second portion widens from the second output facet towards the first output facet. In yet another instance, the second portion is at an angle from the second output facet towards the first output facet in such a manner that the second portion lies straight from the second output facet towards the first output facet. Optionally, a diameter of the second output facet is lesser than a diameter of the first output facet. When the optical attachment is in use, the second output facet receives the light emitted from the one or more light sources. Thus, the posterior facet is arranged in such a manner such that the received light is guided towards the second output facet and the first output facet via the hollow body.

Optionally, the term “second illuminated image” refers to an image of the second output facet formed on the target, when the received light is emitted towards the target from the second output facet. In other words, the second illuminated image of the second output facet is formed on the target when the light received from the one or more light sources is projected through the posterior surface to the target via the second output facet. In this regard, when in use, the second illuminated image represents a circular shape of the second output facet. When the optical attachment is in use, the formation of the second illuminated image clearly is an additional indication of a required position of the optical attachment with respect to the target. As an example, the second illuminated image is a circular ring-shaped image having smaller radius than the first illuminated image (of circular ring shape). A technical benefit of forming the second illuminated image is that second illuminated image is smaller than pupil and the first is larger i.e. the pupil appears between the first and the second images. This way distance measurement is more accurate and alignment is better.

Optionally, the target is a surface of an eye and when the target is at a predefined distance (D) from the first output facet, the first illuminated image, having a second radius, is formed at the surface of the eye, wherein the second radius is 1.0 times up to 1.2 times radius of a pupil of the eye. As an example of radius of the pupil is 4 mm then the radius of the first illuminated image would be 4 mm to 1.2×4=4.8 mm. Second radius could be 1.0, 1.05, 1.1, 1.15 up to 1.05, 1.1, 1.15, 1.2 times radius of pupil of the eye.

Optionally, when the target is an eye and at the predefined distance (D) between a surface of a pupil and the first output facet, the first illuminated image is formed at an outer diameter of the pupil. Herein, the “pupil” is a circular opening in a centre of an iris of the eye. The term “surface of the pupil” refers to an edge or a boundary of this circular opening, at a junction of the iris and the pupil. Herein, the surface of the pupil is a part of the pupil that is visible from a front of the eye. The term “outer diameter” refers to a widest part of the pupil, at the junction of the iris and the pupil.

Optionally when the target is at the predefined distance (D) from the first output facet, the second illuminated image, having a third radius, is formed at the eye, wherein the third radius is 0.5 up to 1.0 times radius of the pupil of the eye. This ensures that a second image is smaller than the pupil providing indication of the distance from that regard.

Optionally, when the first illuminated image is formed at the outer diameter of the pupil and the second illuminated image is formed at the inner diameter of the pupil, a double ring pattern image is formed on the surface of the pupil. A technical benefit of forming the double ring pattern image is to enable a precise alignment of the tip of the eye probe in two-dimensional (2D) coordinates i.e., x-coordinate and γ-coordinate, when the eye is at the predefined distance (D) from the first output facet, hence facilitating the optical attachment to function as a distance meter and an aligner.

Optionally the first radius (r1) is:
r1=−r2×(D−f)/f, wherein r2 the second radius, D is the predetermined distance and f is half of curvature of the eye surface. A surface of an eye can be considered for this disclosure to be a convex mirror having a focal length f. As an example, an average adult eye has an anterior to posterior diameter of approximately 24 millimeters, so its radius would be about 12 mm. This would mean that the focal length (f) of the eye, if treated as a convex mirror, would be roughly half the radius of curvature, or about 6 mm. For a mirror equation (below), the focal length for a convex mirror is considered as a negative value, so f would be −6 mm in this example.

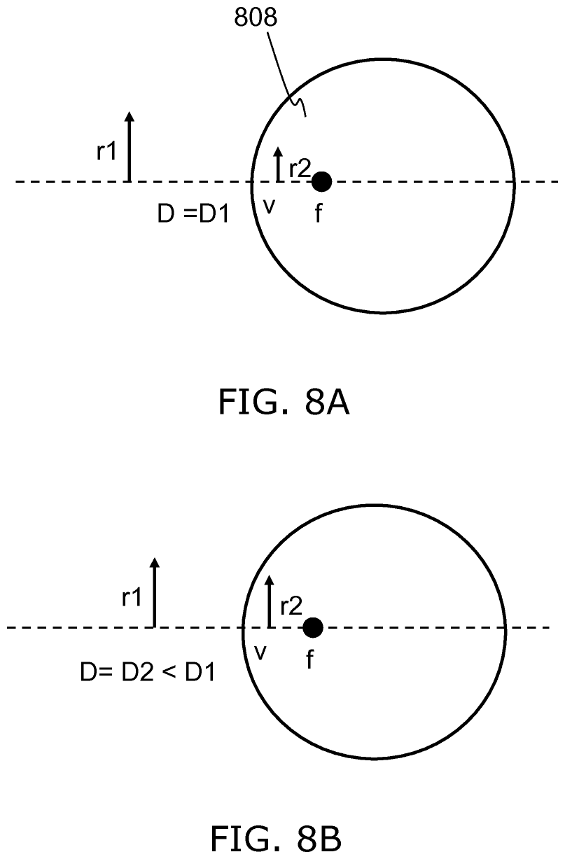

In the mirror equation (see FIGS. 8A and 8B)

1/f=1/v+1/D
f is the focal length, v is the image distance (from the surface of the eyeball towards retina), and D is the distance of a first output facet. In this equation, we can rearrange the mirror equation to get:

v=fD/(D−f)

Further more a magnification equation:

m=−v/D=r2/r1
wherein, r1 is a first radius (i.e. of the first base of the conical frustum of the hollow body i.e. of the first facet if the first facet is circular) of the first facet, r2 is a second radius of the first illuminated image, and m is the magnification.

We can express the magnification m in terms of r2 and r1:

m=r2/r1

Now, we can substitute the value of v from the mirror equation into the magnification equation:

m=−[fD/(D−f)]/D=−f/(D−f)

This gives us the magnification m in terms of the focal length f and the distance D.

To find the first radius r1, we rearrange the magnification equation to solve for r1:

r1=r2/m

Finally, we substitute the new expression for m into this equation, and we get:

r1=r2/[−f/(D−f)]=−r2*(D−f)/f

This is the equation that describes the first radius (r1) of the first output facet as a function of the second radius (r2), the object distance (D), and the focal length (f) of a convex mirror.

The size of the human pupil can change in response to various factors, including light levels, emotional state, age, and certain substances. Regarding light conditions, the diameter of the pupil generally ranges from about 1.5 mm in very bright light to about 8 mm in the dark. The following are approximate values:

In bright light conditions: The pupil size can decrease to around 1.5-2 mm in diameter. This is because the eye needs to limit the amount of light that enters to prevent damage and maintain visual acuity.

In normal light conditions: The pupil size tends to be about 3-4 mm in diameter. This balance allows for both sufficient light intake and focused vision.

In dim light or darkness: The pupil size can expand to approximately 7-8 mm in diameter. The dilation (widening) in low light conditions allows more light to reach the retina, helping to improve night vision

According to embodiment predetermined distance (D) is an operating range of a tonometer from the first output facet to the surface of the eye. In the tonometer a probe is used to obtain (via physical contact) information such as intraocular pressure of an eye. The probe is typically arranged to be movable from a first position to a second position. As an example the probe is initially in the first position (inside of the hollow body) and in the second position tip part of the probe is out side of the probe at predetermined distance D from the first output facet towards the surface of the eye when the tonometer is used.

Optionally, the predefined distance (D) lies in a range of 4 millimetres to 8 millimetres. As an example, the predefined distance (D) may be from 4 or 5 millimetres up to 6, 7 or 8 millimetres. More optionally, the predefined distance (D) lies in a range in a range of 4 millimetres to 7 millimetres. Yet more optionally, the predefined distance (D) lies in a range of 4 millimetres to 6 millimetres. As discussed the D depends on for example type and reach of the probe of the tonometer.

In this regard, the first radius can be calculated using above equations as
r1=−2 mm*(6 mm+6 mm)/(−6 mm)=4 mm if D=6 mm and pupil radius is 2 mm. i.e as design criteria for the first radius 4 mm could be considered. According to an embodiment optical illumination power of the one or more light sources might be controlled to control size of the pupil of an eye of an user. This enables, since the size of the pupil varies according to illumination conditions to control size of the pupil during the measurement. As an example, if the illumination power is set to bright the size of pupil is small. This way we can control size to obtain reference distance on the eye (to which compare size of first or second illuminated image). Control can be done by providing more or less current to LED's (if LED's are used) for example. Term bright refers to “day light type of brightness”. Term dim is equivalent to dark room conditions.

The present disclosure also relates to the method as described above. Various embodiments and variants disclosed above, with respect to the aforementioned optical attachment, apply mutatis mutandis to the method.

According to an aspect a method of determining a distance between an surface of the eye and a tonometer is provided. In said method, an optical attachment is used to provide a first illuminated image of a first output facet of the optical attachment on the surface of the eye. The optical attachment is as discussed above.

Optionally, the method further comprising moving the tonometer towards the surface of the eye and observing a first radius of the first illuminated image in respect to a radius pupil of the eye. Effect of this is that it is easy to visually see (by doctor for example) when the device is in the right distance. Right distance is when the radius of the first illuminated image is approximately same as the radius of the pupil of the eye.

According to an embodiment the tonometer is at right distance when the first radius is 1.0 up to 1.2 times larger in than the radius of the pupil of the eye. Benefit of this is that image is formed at top of iris and it is easy to detect.

Optionally when the tonometer is at the right distance, performing a measurement of an property of the eye with the tonometer. Effect of this is that measurement is done in right distance thus making measurements more reliable.

### DETAILED DESCRIPTION OF THE DRAWINGS

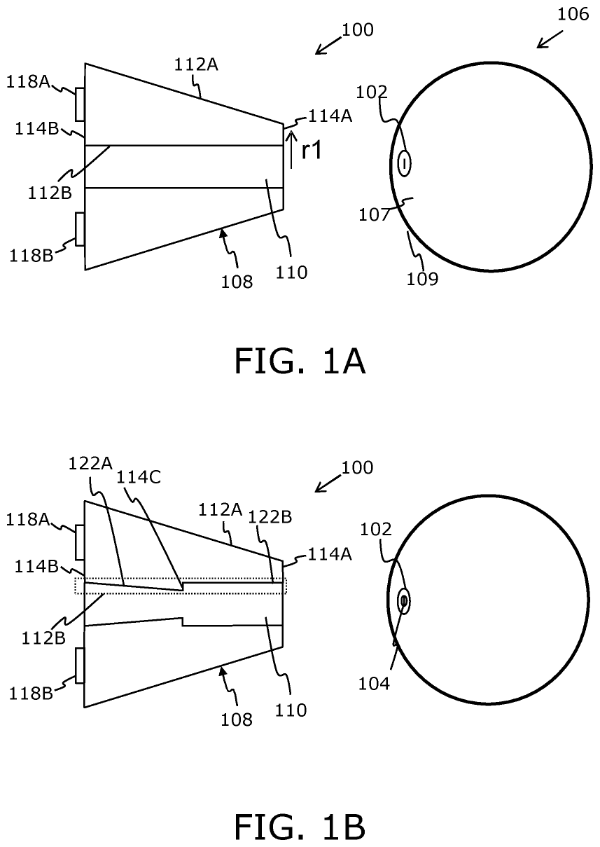

Referring to FIGS. 1A and 1B, there is shown schematic illustrations of formation of a first illuminated image 102 and a second illuminated image 104 of an optical attachment 100 for emitting light towards a target 106, in accordance with different embodiments of the present disclosure. The target 106 is a surface 107 of an eye 109 in the figures. For sake of simplicity, a cross-section view of the optical attachment 100 is shown.

In FIGS. 1A and 1B, the optical attachment 100 comprises a hollow body 108 of light guiding material, the hollow body 108 comprising a through opening 110, an outer surface 112A, an inner surface 112B opposite to the outer surface 112A, a first output facet 114A, and a posterior facet 114B opposite to the first output facet 114A. The posterior facet 114B is arranged to receive light from one or more light sources (denoted by 118A and 118B) and to guide the received light via the hollow body 108 towards the first output facet 114A. When in use, the first output facet 114A is configured to emit the light, 1 towards the target 106 forming the first illuminated image 102 of the first output facet 114A on the target 106. The first output facet has a first radius of r1.

In FIG. 1B, the inner surface 112B includes a stepped configuration defining a first portion 122A, a second portion 122B, and a second output facet 114C between the first portion 122A and the second portion 122B, the second output facet 114C being parallel to the first output facet 114A. Herein, the posterior facet 114B is further arranged to guide the received light via the hollow body 108 towards the second output facet 114C. The second output facet 114C when in use is configured to emit the light towards the target 106 forming the second illuminated image 104 of the second output facet 114C on the target 106.

FIGS. 1A and 1B are merely examples, which should not unduly limit the scope of the claims therein. It is to be understood that the specific implementations of the optical attachment 100 are provided as examples. A person skilled in the art will recognise many variations, alternatives, and modifications of embodiments of the present disclosure.

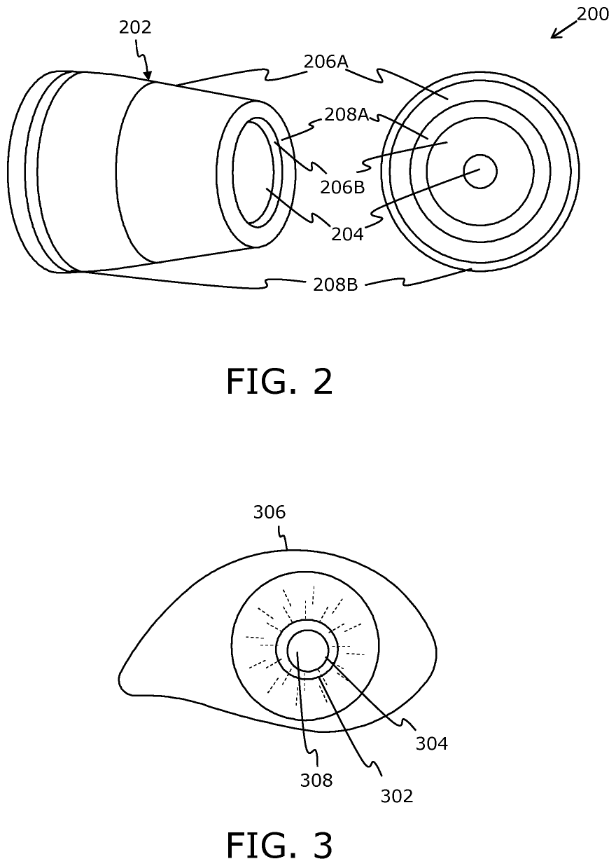

Referring to FIG. 2, there is shown an isometric view and a front view of an optical attachment 200, in accordance with an embodiment of the present disclosure. The optical attachment 200 comprises a hollow body 202 of light guiding material, the hollow body 202 comprising a through opening 204, an outer surface 206A, an inner surface 206B opposite to the outer surface 206A, a first output facet 208A, and a posterior facet 208B opposite to the first output facet 208A.

FIG. 2 is merely an example, which should not unduly limit the scope of the claims therein. It is to be understood that the specific implementations of the optical attachment 200 are provided as examples. A person skilled in the art will recognise many variations, alternatives, and modifications of embodiments of the present disclosure.

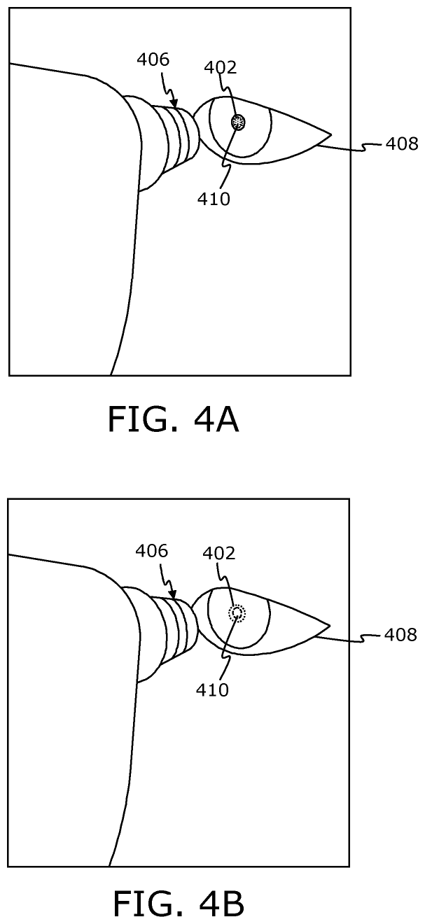

Referring to FIG. 3, there is shown a schematic illustration of formation of a first illuminated image 302 and a second illuminated image 304 on a target, in accordance with an embodiment of the present disclosure. Herein the target is an eye 306. The first illuminated image 302 is formed at an outer diameter of a pupil 308 of the eye 306. The second illuminated image 304 is formed at an inner diameter of the pupil 308 of the eye 306.

FIG. 3 is merely an example, which should not unduly limit the scope of the claims therein. A person skilled in the art will recognise many variations, alternatives, and modifications of embodiments of the present disclosure.

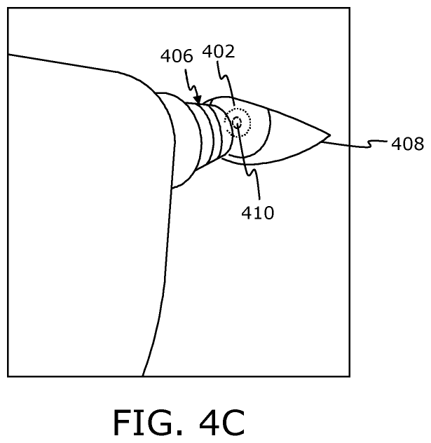

Referring to FIGS. 4A, 4B, and 4C, there are shown exemplary schematic illustrations of formation of a first illuminated image 402 and a second illuminated image 404 on a target using an optical attachment 406, in accordance with an embodiment of the present disclosure. In FIGS. 4A, 4B and 4C, the target is an eye 408. In FIG. 4A, the first illuminated image 402 is unclear i.e., the first illuminated image 402 is smaller than pupil 410 (as shown by a dot-patterned circle) of the eye 408. This due to distance D being too far. o This occurs when a surface of the pupil 410 is not at a predefined distance (D) from a first output facet of the optical attachment 406.

In FIG. 4B, the first illuminated image 402 slightly (1.1 times) larger than the pupil 410. This is due that the optical attachment (output facet of) is at right predefined distance (D) from the surface of the eye.

In FIG. 4C, when the eye 408 is too close i.e closer than predetermined distance (D). As illustrated the first illuminated image 402 is significantly (say 1.5 times radius) larger than pupil 410. This indicates that device is too close.

FIGS. 4A, 4B and 4C are merely examples, which should not unduly limit the scope of the claims therein. It is to be understood that the specific implementations of the optical attachment 406 are provided as examples. A person skilled in the art will recognise many variations, alternatives, and modifications of embodiments of the present disclosure.

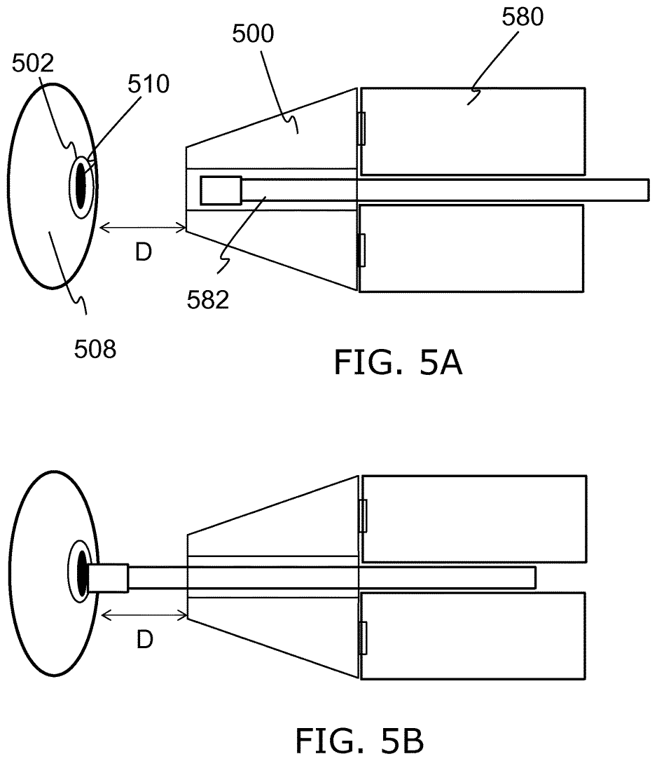

Referring to FIGS. 5A and 5B, there is shown a cross-section view of an optical attachment 500 when attached to a tonometer 580. The tonometer 580 comprises a probe 582. The probe can be used to measure a property of an eye 508. In FIG. 5A is illustrates a situation in which the first output facet of the optical attachment 500 is at a predetermined distance D from a surface of the eye 508. A first illuminated image 502 is formed around the pupil 510 as indicated. The probe 582 is in side of the hollow body of the optical arrangement. Since the distance is right (based on visual indicator) the probe can be moved from inside position to outside (measurement position) as indicated in the FIG. 5B. In the figure the probe is in contact with the surface of the eye and a measurement of the eye property is done. This way it is possible control that the measurement is done at an operating range (reach of the probe in this example) i.e the predetermined distance is operating range.

FIG. 5 is merely an example, which should not unduly limit the scope of the claims therein. A person skilled in the art will recognise many variations, alternatives, and modifications of embodiments of the present disclosure.

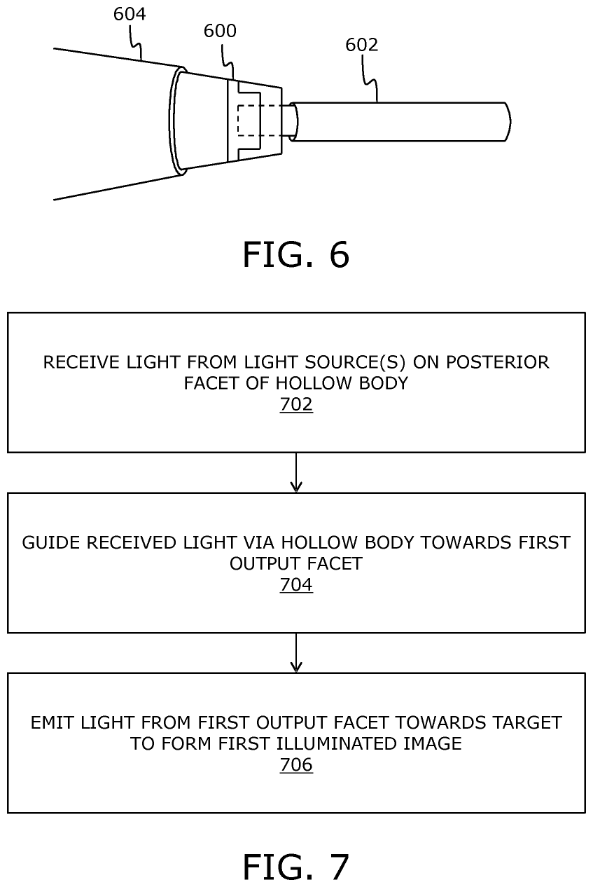

Referring to FIG. 6, there is shown an optical attachment 600 comprising a detachable tube 602, in accordance with an embodiment of the present disclosure. The optical attachment 600 is any one of detachably attached or permanently attached to a tonometer 604. The detachable tube 602 is configured to be accommodated inside a through opening of the optical attachment 600, wherein the detachable tube 602 is configured to accommodate an eye probe for attaching or detaching the eye probe to the tonometer 604.

FIG. 6 is merely an example, which should not unduly limit the scope of the claims therein. A person skilled in the art will recognise many variations, alternatives, and modifications of embodiments of the present disclosure.

Referring to FIG. 7, illustrated is a flowchart depicting steps of a method of emitting light towards a target using an optical attachment comprising a hollow body of a light guiding material, in accordance with an embodiment of the present disclosure. At step 702, light from one or more light sources is received on a posterior facet of the hollow body, the hollow body further comprising a through opening, an outer surface, an inner surface opposite to the outer surface, a first output facet opposite to the posterior output facet. At step 704, the received light via the hollow body is guided towards the first output facet. At step 706, the light from the first output facet is emitted towards the target to form a first illuminated image of the first output facet on the target. FIGS. 8A and 8B are illustrations on how an eye 808 can be considered to be convex mirror type of object. An arrow (this corresponds to radius of the first output facet) having height of a first radius r1 is presented at distance D=D1 in the FIG. 8A. A virtual image (arrow) inside of the eye 808 is formed at distance v from the surface of the eye. The virtual image has height corresponding to second radius r2. Focal point f of the convex mirror (generalization of the eye) is illustrated. The focal point f is approximately ½ of radius of the eye ball. In FIG. 8B it is shown that when the arrow (optical attachment) is closer the image is larger. In FIG. 8B predetermined distance D is D2 which is smaller than D1.

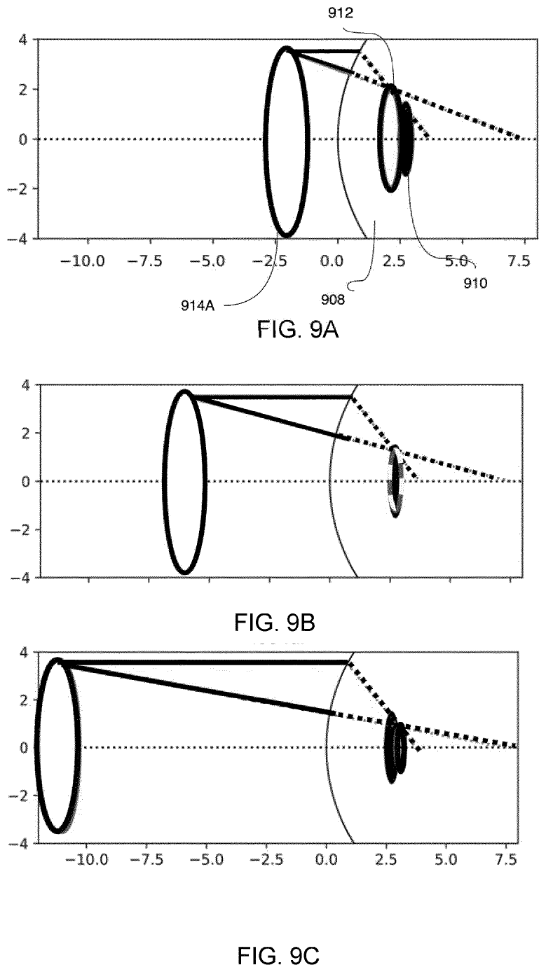

FIG. 9A is an illustration of a situation on which a first output facet 914A which is illuminating light is too close (2.5 mm from) to eye 908. This is indicated as a first illuminated image 912 is larger than pupil 910. Sold line and dashed line represent light rays forming virtual image on the convex mirror like surface of an eye. X axis is distance in millimeters from the surface of eye. In FIG. 9A 2.5 mm from the surface.

In FIG. 9B tonometer, thus the first output facet is at correct (predetermined) distance of about 6 mm. (in the figures the radius of first output facet is about 4 mm. This dimensioning provides, for an adult patient, current size of illuminated image as illustrated in FIG. 9B. This formation of light ring around the pupil has been found a good and reliable indicator for a doctor to operate tonometer. In practice when the doctor sees that the first illuminated image matches size of pupil the tonometer can be operated safely.

FIG. 9C is an illustration of situation on which the tonometer (and thus the first output facet) is too far (more then 10 mm). The first illuminated image is smaller than pupil.

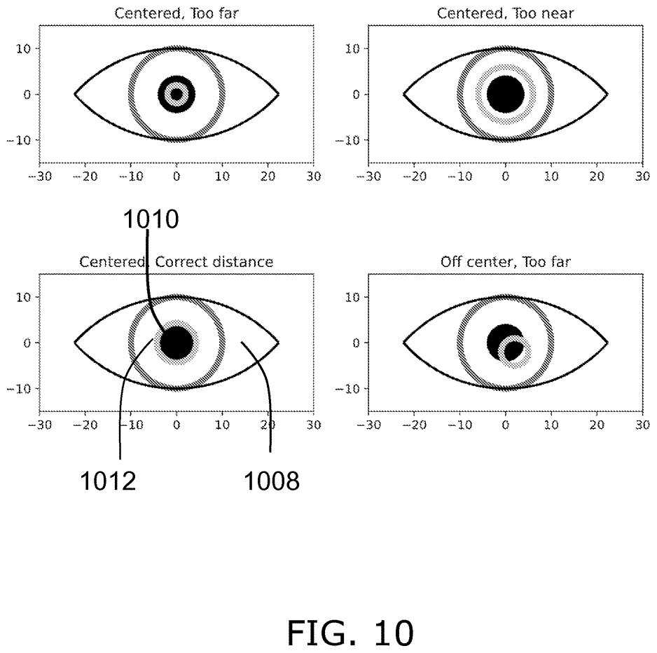

FIG. 10 is an illustration of eye 1008 seen from direction of using a tonometer. The first illuminated image 1010 is drawn with grey scale circle. Pupil 1010 is indicated with solid black circle.

In top left image is illustrated when the tonometer (and thus the first output facet of an apparatus which is attached to the tonometer) is too far. The first image is smaller than the pupil.

In top right image is illustrated when the tonometer (and thus the first output facet of an apparatus which is attached to the tonometer) is too close. The first image is larger than the pupil,

In bottom left image is illustrated when the tonometer (and thus the first output facet of an apparatus which is attached to the tonometer) is at right distance. The first image is same size than the pupil.

In bottom right image is illustrated when the tonometer (and thus the first output facet of an apparatus which is attached to the tonometer) is too far and not centred with the pupil. The first image is smaller than the pupil and does not have same centre as the pupil. Indeed this is additional benefit i.e. if the canter of the first image is aligned with centre of pupil the alignment is right.

The aforementioned steps are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. An optical attachment for emitting light towards a target, the optical attachment comprising:
a hollow body of light guiding material, the hollow body comprising a through opening, an outer surface, an inner surface opposite to the outer surface, a first output facet, and a posterior facet opposite to the first output facet; and
the posterior facet arranged to receive light from one or more light sources and to guide the received light via the hollow body towards the first output facet; and
wherein the first output facet when in use is configured to emit the light towards the target forming a first illuminated image of the first output facet on the target.

2. An optical attachment according to claim 1, wherein the hollow body is shaped as a conical frustum of a cylinder, wherein the first output facet forms a first base of the conical frustum, the posterior facet forms a second base the conical frustum and the first base of the conical frustum has a first radius.

3. An optical attachment according to claim 1, wherein the inner surface includes a stepped configuration defining a first portion, a second portion, and a second output facet between the first portion and the second portion, the second output facet being parallel to the first output facet, and
wherein the posterior facet is further arranged to guide the received light via the hollow body towards the second output facet, and
wherein the second output facet when in use is configured to emit the light towards the target forming a second illuminated image of the second output facet on the target.

4. An optical attachment according to claim 1, wherein when the target is a surface of an eye and when the target is at a predefined distance (D) from the first output facet, the first illuminated image, having a second radius, is formed at the surface of the eye radius, wherein the second radius is 1.0 times up to 1.2 times radius of a pupil of the eye.

5. An optical attachment according to claim 3, wherein when the target is at the predefined distance (D) from the first output facet, the second illuminated image, having a third radius, is formed at the eye, wherein the third radius is 0.5 up to 1.0 times radius of the pupil of the eye.

6. An optical attachment according to claim 4 wherein the predetermined distance (D) is an operating range of a tonometer from the first output facet to the surface of the eye.

7. An optical attachment according to claim 2, wherein the first radius (r1) is:
r1=−r2×(D−f)/f, wherein r2 the second radius, D is the predetermined distance and f is half of curvature of the eye surface.

8. An optical attachment according to claim 3, wherein the predefined distance (D) lies in a range of 4 millimetres to 8 millimetres.

9. An optical attachment according to claim 3, wherein the first radius is between 3 millimetres to 10 millimetres.

10. An optical attachment according to claim 1, wherein the hollow body is configured to surround an eye probe comprised in the tonometer.

11. An optical attachment according to claim 10, further comprising a detachable tube configured to be accommodated inside the through opening, wherein the detachable tube is configured to accommodate the eye probe for attaching or detaching the eye probe to the tonometer.

12. An optical attachment according to claim 1, wherein the one or more light sources are operable to generate stray light that obscures visibility of the eye probe during measurement of at least one property of the eye.

13. An optical attachment according to claim 1 where in illumination power of the one or more light sources is controlled to control size of pupil of an eye of user.

14. A method of determining a distance between a surface of the eye and a tonometer, the method comprising using an optical attachment according to claim 1 to provide a first illuminated image of a first output facet of the optical attachment on the surface of the eye.

15. A method according to claim 14, wherein the method further comprising moving the tonometer towards the surface of the eye and observing a first radius of the first illuminated image in respect to a radius pupil of the eye.

16. A method according to claim 15, wherein the tonometer is at right distance when the first radius is 1.0 up to 1.2 times larger in than the radius of the pupil of the eye.

17. A method according to claim 16, wherein when the tonometer is at the right distance, performing a measurement of an property of the eye with the tonometer.
